(19) United States
(12) Patent Application Publication
(10) Pub. No.: US 2017/0252470 A1
(43) Pub. Date: Sep. 7, 2017

(54) Sanitizing Biogenic Waste Using Waste Gas from an Incineration System

(71) Applicant: Entrade Energiesysteme AG, Duesseldorf (DE)

(72) Inventors: Stefan Aniol, Munich (DE); Martin Oelkers, Roehrmoos (DE); Julien Uhlig, Dahlem (DE)

(21) Appl. No.: 15/499,825

(22) Filed: Apr. 27, 2017

Related U.S. Application Data
(63) Continuation-in-part of application No. PCT/EP2015/074834, filed on Oct. 27, 2015.

(30) Foreign Application Priority Data
Oct. 27, 2014 (DE) 102014221846.2
Oct. 27, 2015 (EP) PCT/EP2015/074834

(51) Int. Cl. A61L 2/06; F23B 80/00; F23G 5/46; B09B 3/00; A61L 2/26

(52) U.S. Cl. CPC A61L 2/06; B09B 3/0083; A61L 2/26; F23G 5/46; F23B 80/00; A61L 2202/11; A61L 2202/14

(57) ABSTRACT

In newly industrialized countries and Third World countries, biogenic waste frequently constitutes a threat to ground water due to its being contaminated with bacteria and germs. In order to decontaminate such biogenic waste, one or more closable sanitizing chambers are fitted with open containers that are filled with the biogenic waste. The sanitizing chambers are closed, and hot waste gases (80° C. to 140° C.) are guided through the sanitizing chambers until germs and bacteria in the biogenic waste are killed. The sanitizing of biogenic waste eliminates the waste as a source of diseases and ground water contamination. The invention is suitable for decentralized locations, for example, in Third World countries or newly industrialized countries or places with stand-alone grids. The invention can have either a stationary or a mobile design.

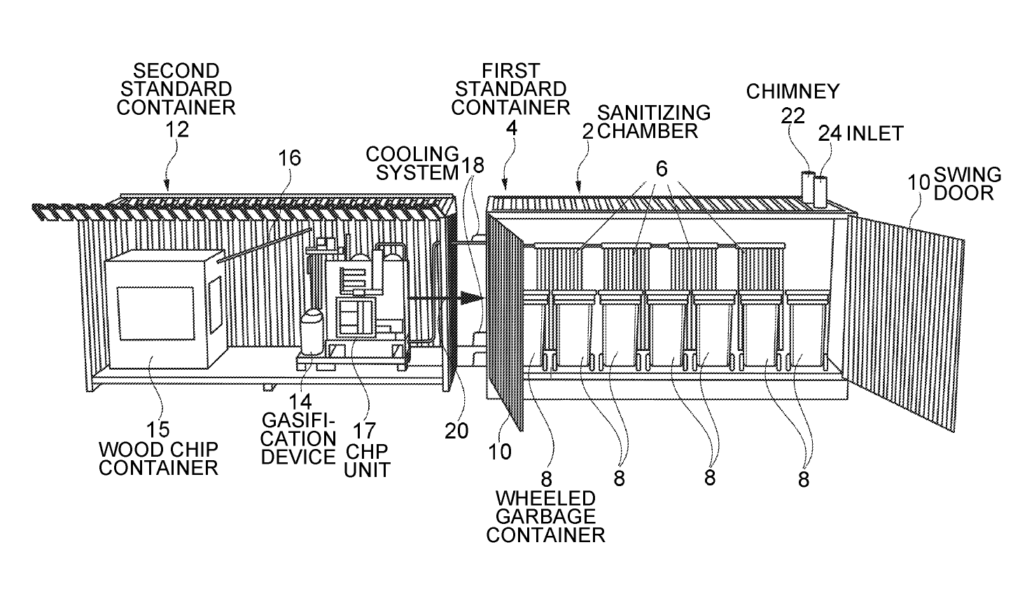

### CROSS REFERENCE TO RELATED APPLICATION

This application is filed under 35 U.S.C. §111(a) and is based on and hereby claims priority under 35 U.S.C. §120 and §365(c) from International Application No. PCT/EP2015/074834, filed on Oct. 27, 2015, and published as WO 2016/066626 A2 on May 6, 2016, which in turn claims priority from German Application No. 102014221846.2, filed in Germany on Oct. 27, 2014. This application is a continuation-in-part of International Application No. PCT/EP2015/074834, which is a continuation of German Application No. 102014221846.2. International Application No. PCT/EP2015/074834 is pending as of the filing date of this application, and the United States is an elected state in International Application No. PCT/EP2015/074834. This application claims the benefit under 35 U.S.C. §119 from German Application No. 102014221846.2. The disclosure of each of the foregoing documents is incorporated herein by reference.

### TECHNICAL FIELD

The invention relates to a method for sanitizing biogenic wastes with waste gases from incineration device and to a device for carrying out the method.

### BACKGROUND

In newly industrialized countries and Third World countries, biogenic waste frequently constitutes a threat to ground water due to its being contaminated with bacteria and germs. Biogenic waste is composed mainly of organic matter, such as manure, sewage, sawdust, food scraps and agricultural waste. Biogenic waste is also called biohazard infectious waste or simply biowaste. It is therefore an object of the present invention to provide a simple method for the hygienization or sanitizing of biowaste that can be performed without much technical effort, as well as an apparatus for performing the method.

### SUMMARY

The invention relates to a method for sanitizing biowaste using waste heat and the toxicity and high temperature of waste gas generates in a combustion system, such as a combined heat and power (CHP) unit. In one embodiment, the combustion system includes a combustion engine that turns an electric generator. The biowaste is introduced into a sanitizing chamber in plastic, wheeled garbage container that are open. Waste gas is generated in the combustion system. The waste gas has a temperature greater than 80° C. and a concentration of carbon dioxide greater than that of ambient air. The waste heat is also generated in the combustion system. The waste heat is transferred in a fluid that flows through a cooling system. The waste gas flows through the sanitizing chamber until germs and bacteria contained in the biowaste are killed by the toxicity and high temperature of waste gas. The waste heat is transferred via the fluid into the sanitizing chamber. The germs and bacteria in the biowaste are also killed by the high temperature in the sanitizing chamber caused by transferring the waste heat from the combustion system through the cooling system and into the sanitizing chamber. The cooling system is connected to a heat radiator in the sanitizing chamber. The waste heat in the fluid flowing through the cooling system is transferred through the heat radiator and into the sanitizing chamber.

An apparatus for sanitizing biowaste includes one or more sanitizing chambers, a combustion system, a cooling system, a waste gas pipe and a gas outlet. In one embodiment, each sanitizing chamber is contained in a standard metal shipping container. The sanitizing chamber has a door through which a wheeled, hard-plastic garbage container can be placed in the chamber. The garbage container is open on the top and contains biohazard infectious waste. In one embodiment, the combustion system is an incineration system in which agricultural or forestry refuse is burned. In another embodiment, the combustion system is a combined heat and power (CHP) unit that runs on wood gas generated from wood chips. The combustion system generates waste gas that has a temperature greater than 80° C. and a concentration of carbon dioxide greater than ambient air. The combustion system also generates waste heat. The cooling system contains a fluid through which the waste heat is transferred away from the combustion system. The fluid flowing through the cooling system transfers the waste heat from the combustion system into the sanitizing chamber. A heat radiator is disposed inside the sanitizing chamber. The fluid flows through the heat radiator, and the waste heat in the fluid is transferred through the heat radiator and into the sanitizing chamber. The waste gas flows from the combustion system through the waste gas pipe into the sanitizing chamber and out the gas outlet. The germs and bacteria contained in the biowaste are killed by the waste gas flowing through the sanitizing chamber and by the waste heat transferred into the sanitizing chamber.

Other embodiments and advantages are described in the detailed description below. This summary does not purport to define the invention. The invention is defined by the claims.

### BRIEF DESCRIPTION OF THE DRAWING

The accompanying drawing illustrates embodiments of the invention.

The figure is a schematic diagram of an embodiment of the invention showing a sanitizing chamber in the form of a standard container.

### DETAILED DESCRIPTION

Reference will now be made in detail to some embodiments of the invention, examples of which are illustrated in the accompanying drawing.

The figure shows an exemplary configuration of the invention with a sanitizing or hygienization chamber 2 in the form of a first standard container 4. The first standard container 4 and a second standard container 12 resemble metal shipping containers. In one embodiment, the sanitizing chamber 2 is contained in a standard 40-foot metal shipping container. A plurality of heat radiators 6 are arranged on the walls inside the standard container 4. A plurality of wheeled garbage containers 8 are also disposed inside the first standard container 4. Open garbage containers 8 are loaded with the biowaste that will be sanitized, decontaminated and hygienized. The sanitizing chamber 2 (first standard container 4) is provided with swing doors 10 that allow the garbage containers 8 to be loaded and unloaded from the long side as opposed to the end of the container.

A device 14 for the gasification of wood is disposed in the second standard container 12. The gasification device 14 is fueled with wood chips or pellets from which wood gas is generated. The wood chips or pellets are transferred from a chip or pellet container 15 through a conduit 16 into the gasification device 14. The wood gas generated by the gasification device 14 is used to power an engine of a combined heat and power (CHP) unit 17. The engine of the CHP unit 17 typically runs a generator that generates electricity. The cooling system 18 for the engine also generates “waste heat” that can be used for heating in cooler climates as well as for sanitizing the biowaste. The cooling system 18 of the engine of the CHP unit 17 is connected to the heat radiators 6 in the sanitizing chamber 2. The cooling system 18 is operated with water that exits the engine under pressure at a temperature of about 200° C. The hot water exiting the CHP unit 17 is pre-cooled in the length of the pipes of the cooling system 18 outside the second standard container 12 to a temperature in the range of 90° C. to 130° C. before the heated water enters the heat radiators 6. Water at the temperature exiting the CHP unit 17 would damage the sanitizing chamber 2. Water cooler than 90° C. is then returned through the cooling system back to the CHP unit 17. Thus, waste heat from the CHP unit 17 is transferred into the sanitizing chamber 2. In addition, hot waste gas from the CHP unit 17 and the gasification device 14 is guided through a waste gas pipe 20 into the sanitizing chamber 2. The waste gas is exhaust gas from the engine. The first standard container 4 (sanitizing chamber 2) is provided with a chimney 22 that allows waste gas to escape from the sanitizing chamber 2 into the ambient air. Fresh air can be pumped into the sanitizing chamber 2 through a fresh air inlet 24.

In operation, biowaste is loaded into the wheeled garbage containers 8, and the loaded garbage containers 8 are placed into the sanitizing chamber 2 through the open swing doors 10. The swing doors 10 are then closed, and waste heat from the CHP unit 17 and gasification device 14 is transferred to the sanitizing chamber 2 via the cooling system 18 and radiates from heat radiators 6 into the sanitizing chamber 2. Simultaneously, waste gas from the CHP unit 17 and gasification device 14 is transferred to the sanitizing chamber 2 via the waste gas pipe 20. The garbage containers 8 are open in order to allow vapor to exit from the biowaste during the drying process. The waste gas and vapor exits via the waste gas chimney 22 into the ambient air. Due to the combined action of the waste heat and hot waste gas, the biowaste in the garbage containers 8 is decontaminated and dried. The decontamination kills the germs and bacteria. The contents of the garbage containers 8 is no longer biohazard infectious waste. After being sanitized, the biowaste may be composted, fermented or directly used for improving the soil without the danger of contaminating the environment. Before unloading the garbage containers 8 with the decontaminated and dried biowaste from the sanitizing chamber 2, the waste gas pipe 20 is decoupled from the sanitizing chamber 2, and the sanitizing chamber 2 is flooded with fresh air through the fresh air inlet 24 in order to flush out toxic waste gas from the sanitizing chamber 2. Then the swing doors 10 can be opened without the danger to the user of suffocating, and the garbage containers 8 can be unloaded. The sanitizing process can be started again with new biowaste loaded into the garbage containers 8.

More than one sanitizing chamber 2 can be connected to one second standard container 4 with gasification device 14 and CHP unit 17, thus allowing batch processing of the plurality of sanitizing chambers 2.

One or more closable sanitizing chambers 2 are loaded with open containers 8 filled with biowaste. The sanitizing chamber 2 is closed, and hot waste gases at a temperature of 80° C. to 140° C. are guided via the waste gas pipe 20 through the sanitizing chamber(s) until germs and bacteria in the biowaste are killed.

The waste heat from the combustion system is introduced into the at least one sanitizing chamber 2 through the gas circuit in addition to the heat from the engine cooling system 18 that is transferred to the sanitizing chamber through the fluid circuit. Both sources of heat are used to kill germs and bacteria in the biowaste.

Through the hygienization of the biowaste, the waste is eliminated as a source of diseases and ground water contamination. The invention is suited for decentralized locations, such as are common in Third World countries and newly industrialized countries, and in places with stand-alone grids. The biowaste sanitizing system can be configured as either a stationary and mobile system.

In the event that the temperature in the sanitizing chamber 2 exceeds a critical threshold, the waste gas from waste gas pipe 20 is guided directly into the chimney 22. The control of the shunting of the waste gas when the temperature exceeds the threshold is performed automatically or manually.

The sanitizing chambers 2 are protected against explosion by rendering them inert with waste gas that contains a high concentration of carbon dioxide (CO₂). The waste gas thereby prevents the explosive gas methane (CH₄), which is generated during the fermentation of the biowaste, from coming in contact with oxygen in the hot sanitizing chamber 2. The methane is flushed out of the sanitizing chamber 2 through the chimney 22 by the waste gas flowing through the chamber.

Biowaste is put into the sanitizing chambers 2 in standard wheeled plastic garbage containers 8, such as those commonly collected at the street in residential neighborhoods. By loading the biowaste into the chambers 2 in standard garbage containers 8, loose biowaste need not be handled. Moreover, no wheeled loader or other machines are required to load the biowaste into the chambers 2.

Water in addition to that flowing in the cooling system 18 is not required to improve the heat transfer from the CHP unit 17 to the sanitizing chamber 2. The time during which the biowaste is retained in the sanitizing chamber 2 is adjusted as needed.

Before being filled with biowaste, the chamber 2 is open and free of waste gases. Prior to opening, the chamber 2 is flushed with ambient air. In addition, gas sensors for carbon monoxide and carbon dioxide are used as an alerting means to prevent exposure of the users of the biowaste sanitizing system to any poisonous and suffocating atmosphere.

The sanitized biowaste output from the system has a higher dry matter content than the contaminated biowaste because of the heat input. The output biowaste is slightly dried, which improves transportability. This is especially of advantage in areas with difficult road conditions. After being sanitized, the biowaste can be composted, fermented or used directly for improving the soil.

The biowaste sanitizing system has no moving parts other than the engine used to turn the electric generator. Thus, the system requires fewer repairs. Only the waste containers 8 are moved. The use of the hot waste gas was well as the hot water from the engine cooling system 18 to sanitize the biowaste improves the overall efficiency of the sanitizing system.

There are various alternative embodiments for the source of the hot waste gas. The waste gas can be sourced from (i) a combined heat and power unit (CHP) powered using biomass gasification, (ii) a combined heat and power unit (CHP) powered by natural gas (methane), (iii) diesel engines and generators, or (iv) other combustion systems and heaters. In each case, the waste heat in excess of 70° C. is used for sanitizing, and waste gas with a low oxygen content is used for flushing and making the sanitizing chamber 2 inert to explosions.

The sanitizing chambers and second containers can be stand alone or internally divided containers. The first and second containers 4, 12 can also be realized as rooms or cabinets of a building. Each sanitizing chamber 2 is connected to a waste gas supply line 20, a further heat source from the cooling system 18 of the CHP unit 17 and a discharge means for the waste gas, such as through a chimney 22. The sanitizing chamber 2 is sealed with respect to the second container or the other rooms of a building. The sanitizing chamber 2 includes safety technology to guard against the unauthorized entry of the chamber and the exposure of users to any poisonous and suffocating atmosphere. The alerting means for carbon monoxide and dioxide has a blinking light and horn. The sanitizing chamber 2 also has an indicator confirming that the doors 10 are completely closed.

By using additional sources and amounts of waste gas, the biowaste sanitizing system can also be used to achieve an even lower moisture content of the output biowaste. Drier biowaste is lighter and easier to transport.

### REFERENCE NUMERALS

2 sanitizing chamber

4 first standard container

6 heat radiators

8 garbage containers with wheels

10 swing doors

12 second standard container

14 gasification device

15 wood chip or pellet container

16 conduit

17 combined heat and power (CHP) unit

18 cooling system of CHP unit

20 waste gas pipe

22 waste gas chimney

24 fresh air inlet

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-14. (canceled)

15. A method for sanitizing biowaste, comprising:
introducing biowaste into a sanitizing chamber;
generating waste gas in a combustion system, wherein the waste gas has a temperature greater than 80° C. and a concentration of carbon dioxide greater than ambient air;
generating waste heat in the combustion system, wherein the waste heat is transferred in a fluid flowing through a cooling system;
flowing the waste gas through the sanitizing chamber until germs and bacteria contained in the biowaste are killed; and
transferring the waste heat in the fluid through the cooling system and into the sanitizing chamber.

16. The method of claim 15, wherein the germs and bacteria contained in the biowaste are killed by the toxicity and high temperature of the waste gas.

17. The method of claim 15, wherein the combustion system is an engine that turns an electric generator.

18. The method of claim 15, further comprising:
flushing the sanitizing chamber with fresh air after flowing the waste gas through the sanitizing chamber; and
opening the sanitizing chamber and removing the biowaste.

19. The method of claim 15, further comprising:
cooling the fluid flowing through the cooling system to a temperature within a range of 90° C. to 130° C. before the fluid enters the sanitizing chamber.

20. The method of claim 15, further comprising:
cooling the waste gas generated in the combustion system to a temperature within a range of 80° C. to 140° C. before the waste gas flows through the sanitizing chamber.

21. The method of claim 15, wherein the biowaste is introduced into the sanitizing chamber in open containers.

22. The method of claim 15, further comprising:
introducing biowaste into a second sanitizing chamber, wherein the waste gas and the fluid containing the waste heat flow through the second sanitizing chamber; and
operating the second sanitizing chamber and the first sanitizing chamber consecutively in a batch mode.

23. The method of claim 15, wherein the sanitizing chamber is a metal shipping container that can be moved.

24. The method of claim 15, wherein the waste gas and the fluid containing the waste heat flow through the sanitizing chamber until the biowaste reached a predetermined drying state.

25. An apparatus for sanitizing biowaste, comprising:
a sanitizing chamber with a door, wherein the sanitizing chamber is adapted to house a wheeled garbage container containing the biowaste;
a combustion system that generates waste gas, wherein the waste gas has a temperature greater than 80° C. and a concentration of carbon dioxide greater than ambient air, wherein the combustion system also generates waste heat;
a cooling system containing a fluid, wherein the waste heat is transferred by the fluid flowing through the cooling system;
a waste gas pipe; and
a gas outlet, wherein the waste gas flows from the combustion system through the waste gas pipe into the sanitizing chamber and out the gas outlet, and wherein the fluid flowing through the cooling system transfers the waste heat from the combustion system into the sanitizing chamber.

26. The apparatus of claim 25, wherein germs and bacteria contained in the biowaste are killed by the waste gas flowing through the sanitizing chamber and the waste heat transferred into the sanitizing chamber.

27. The apparatus of claim 25, wherein a heat radiator is disposed inside the sanitizing chamber, wherein the fluid flows through the heat radiator, and wherein the waste heat in the fluid is transferred through the heat radiator and into the sanitizing chamber.

28. The apparatus of claim 25, wherein the combustion system is a combustion engine that turns an electric generator.

29. The apparatus of claim 25, wherein the combustion system is a combustion engine is a combined heat and power (CHP) unit.

30. The apparatus of claim 29, further comprising:
a gasification device that generates wood gas from wood chips, wherein the wood gas is used to power the CHP unit.

31. The apparatus of claim 25, wherein the sanitizing chamber is contained in a standard metal shipping container.

32. The apparatus of claim 29, further comprising:
a gas sensor that measures the concentration of carbon monoxide in the sanitizing chamber.
